(19) United States
(12) Patent Application Publication
(10) Pub. No.: US 2003/0013082 A1
(43) Pub. Date: Jan. 16, 2003

(54) Khmer angkor natural herb treatment for HIV infected and AIDS disease

(76) Inventors: Ngan Kiem, Salt Lake City, UT (US); Richard S. Kim, San Jose, CA (US)

Correspondence Address: Ngan Kiem, 625 West 400 North, Salt Lake City, UT, 84116, US

(21) Appl. No.: 09/901,156

(22) Filed: Jul. 10, 2001

(51) Int. Cl. C12Q 1/70; G01N 33/53; A61K 39/21

(52) U.S. Cl. 435/5; 435/7.1; 424/208.1

(57) ABSTRACT

The present invention relates to compounds of Natural Herbal and peptide components. The supplement preferably includes the compositions of the present invention are derived from various Chinese herbal medicines or medicinal plants which have a long history of human consumption. Compositions according to the invention have also exhibited in vitro antiviral activities against marine leukemia virus (MuLV) and human immunodeficiency virus (HIV). The process invention provides methods of monitoring and helping of HIV infected and AIDS diseases through the use of ingredient natural herbal and peptide components.

### TECHNICAL FIELD OF THE INVENTION

The field of the present invention is treating HIV infections and AIDS disease. The process of invention pertains to determine the effectiveness of natural herbal supplement treatment of HIV infected and AIDS disease.

### BACK GROUND OF THE INVENTION

The Human Immunodeficiency Virus (HIV) and Acquired Immune Deficiency Syndrome (AIDS).The world has been to believe that a virus (HIV) destroys the human body immune system by killing the white blood cells known as T-cells. The virus continues to reproduce while the immune response system continues to weaken, because antibodies specifically produced against one glycoprotein may be useless against another. Therefore, it is reducing the number of antibodies available to fight the virus.

Despite the rapid growth of research into HIV no medicine against it is yet available. It just delay the HIV process, to keep the patients can live few years longer. Currently,there are three classes of medication called antiretroviral therapy have been approve by the Food and Drug Administration: analog reverse transcriptase inhibitors, non-nucleoside reverse transcriptase inhibitors, and protease inhibitors all work to keep the virus from reproducing, while the virus can reproduce up to ten billion copies of itself daily.

The maintenance of resistance to mycobacteria is an active immunological process, which is compromised by HIV infection. Ever since human immunodeficiency virus type 1 (HIV) was identified as the causative agent of acquired immunodeficiency syndrome (AIDS), investigators have tried to elucidate the factors that determine the course of HIV infection. In 1987, it was first reported that many HIV-infected persons have virus-specific CTLs, and that these cells are present at unprecedented levels compared to those in other chronic viral infections. Progression to disease occurs in almost all infected persons. This stage is marked by a rise in viremia and a steady decline in number of CD4 lymphocytes. Because HIV replicates in activated CD4 cells, there may be a functional deletion of HIV-specific CD4 cells during primary infection.

The method of treating a patient infected with HIV, including an individual that is asymptomatic but tests positive for the HIV antigen, an individual who is symptomatically sick but does not have AIDS related diseases and an individual infected with the HIV virus who has AIDS related diseases.

The mixtures of peptides of the present invention are not only useful in the determination of antibodies against HIV virus, but also for the determination of the AIDS virus since these peptides either free, polymerized or conjugated to an appropriate carrier are useful in eliciting antibodies, in particular monoclonal antibodies, against AIDS virus.

### THE PRESENT OF THE INVENTION

One of the Temple's Khmer Krom has a lot of Book of Khmer Doctrine. No one pay-attention, about the books on the shelf stayed for long, long time ago. In 1998, one monk, his name Kim Sovann known as Thanh Cong has found a herbal guide book how to treat a different sickness. Especially, it has a formula treatment for HIV and AIDS diseases. Until 1999, Ngan Kiem and Richard Kim went to Cambodia do experiment and, tested the herbal with three patients with HIV infected. The result, after one week, patient can eat freely, sleep well and, gain more energy. Nowadays, those patients go to work normal. Therefore, we feel strongly believe that this formula is very effective.

### SUMMARY OF THE INVENTION

In accordance with the present invention, there are provided of a formula of natural herbal supplemental and peptides, which are particularly adapted for detecting 100% of HIV and AIDS. More specifically, the formula of peptides and natural herbal of the present invention are selected from represent #2 and #3 groups which attaches to form of the ingredients of the mixtures of purified Natural Herbal and Peptides components mixes together.

We claim:

1. A process for determining the effectiveness of using natural herbal and peptide components for HIV infections and AIDS disease related, the process comprising the steps of:
a) Detecting the level of transcription active HIV in monocytes to an antibody, wherein the oligonucleotide probe.
b) Comparing the detected HIV levels; and
c) Correlating changes in HIV levels over time with the use of natural herbal supplement.

2. A purified natural herbal supplemental having the formula step of:
Wherein:
a) Bitter Melon Seeds (blend until become powder) amount of 1.5 gram.
b) Sear Mint or Mentha, amount of 2.5 grain.
c) Solenostemon amboinicus, amount of 3 gram.

3. A purified compound of comprising the following peptides:
a) Mud Fish's gall bladder (4 to 5).
b) Python's gall bladder (1 to 2).
Wherein:
a) A represents the #2 group which attaches to form of the extracts without changing its antigenic properties.
b) A represents the #3 group which attaches to form of the mixtures to make peptide more useful without changing its antigenic properties.
c) A represents the #2 and #3 groups which attaches to form of the ingredients of the mixtures of purified Natural Herbal and Peptides components mixes together.
